(19) United States
(12) Patent Application Publication
(10) Pub. No.: US 2023/0136282 A1
(43) Pub. Date: May 4, 2023

(54) THERMOFORMED MEDICAL SYRINGE

(71) Applicant: Conrad A. Flick, III, Bettendorf, IA (US)

(72) Inventor: Conrad A. Flick, III, Bettendorf, IA (US)

(21) Appl. No.: 17/452,912

(22) Filed: Oct. 29, 2021

(51) Int. Cl. A61M 5/00; A61M 5/31; A61M 5/315; A61M 5/32

(52) U.S. Cl. CPC A61M 5/002; A61M 5/3134; A61M 5/31513; A61M 5/3202; A61M 2207/00; A61M 2210/12; A61M 2205/0216; A61M 2205/583; A61M 2005/31506

(57) ABSTRACT

A single use syringe is disclosed that can be manufactured with minimal time and expense. The barrel is, in one example, compressive thermoformed by extruded tubing and with a resilient wall. The plunger can also be thermoformed. A fluid seal can be established between the resilient barrel sidewall and a rigid plunger sealing ring. The open, proximal end of the barrel is thermally flanged to maintain the barrel's configuration and provide a flange for gripping. The syringe can have non-circular cross sectional shapes, multiple lumens and multiple indentations to provide for a wide range of specific uses.

### DRAWINGS

Other objects and advantages of the present invention will become apparent from the following detailed description of the preferred embodiments thereof taken in conjunction with the accompanying drawings wherein:

FIG. 1 is an axonometric view of a syringe body in accordance with one embodiment of the invention;

FIG. 2 is an axonometric view of the thermoformed syringe of FIG. 1 with a sheath in place;

FIG. 3 is a longitudinal, cross-sectional, side view of the thermoformed syringe of FIG. 2;

FIG. 4 is an axonometric view of a thermoformed syringe showing the absence of the distension without inserted plunger, and without the sheath;

FIG. 5 is a side, axonometric view of a thermoformed sheath;

FIG. 6 is a longitudinal cross-sectional, side view of the sheath's engagement indentions;

FIG. 7 is an axonometric view of a syringe's plunger;

FIG. 8 is a traverse, cross-sectional view of the tubular plunger shaft with lateral wings;

FIG. 9 is a longitudinal, cross-sectional view of the tubular plunger shaft with lateral wings;

FIG. 10 is an axonometric view of a syringe in accordance with an alternative embodiment of the invention;

FIG. 11 is an axonometric view of the thermoformed syringe of FIG. 10 with a sheath in place;

FIG. 12 is a longitudinal cross-sectional, side view of the thermoformed syringe of FIG. 11;

FIG. 13 is an axonometric view of a thermoformed syringe showing the absence of the distension without inserted plunger, and without the sheath;

FIG. 14 is an axonometric, side view of a syringe sheath with side apertures;

FIG. 15 is an axonometric view of an injection molded plunger;

FIG. 16 is a detail cross-sectional view showing the syringe plunger in FIG. 15's sealing interface;

FIG. 17 is an axonometric view of a single axis sealed syringe with a domed sealed end wall in accordance with an alternative embodiment of the invention;

FIG. 18 is an axonometric view of the syringe in FIG. 17 with its sheath in place;

FIG. 19 is a side, longitudinal cross-sectional, side view of the thermoformed syringe of FIG. 18;

FIG. 20 is an axonometric view of a thermoformed syringe showing the absence of distention without inserted plunger, and without the sheath;

FIG. 21 is an axonometric, side view of the thermoformed sheath of FIG. 18;

FIG. 22 is a longitudinal cross-sectional, side view of the sheath revealing its engagement indentions;

FIG. 23 is an axonometric view of an injection molded plunger with domed distal surface;

FIG. 24 is a traverse, cross-sectional view of the molded plunger shaft revealing its seal interface;

FIG. 25 is an axonometric view of a thermoformed syringe with conical end wall showing the absence of distention without inserted plunger, and without the sheath;

FIG. 26 is a side, longitudinal cross-sectional, side view of the thermoformed syringe of FIG. 25;

FIG. 27 is an axonometric view of an injection molded plunger with conical distal surface;

FIG. 28 is an axonometric view of an indention sealed syringe in accordance with an alternative embodiment of the invention;

FIG. 29 is an axonometric view of the thermoformed syringe of FIG. 25 with a sheath in place;

FIG. 30 is a longitudinal cross-sectional, side view of the thermoformed syringe of FIG. 29;

FIG. 31 is an axonometric view of a thermoformed syringe showing the absence of distensions without inserted plunger, and without its sheath;

FIG. 32 is an axonometric, side view of a syringe sheath;

FIG. 33 is an axonometric view of an extruded, tubular plunger with optional cap;

FIG. 34 is a longitudinal, cross-sectional view of the syringe plunger in FIG. 30 showing its sealing interface;

FIG. 35 is an axonometric view of a syringe with integral sheath in accordance with an alternative embodiment of the invention with an integral sheath;

FIG. 36 is a longitudinal, cross-sectional, side view of the thermoformed syringe of FIG. 35;

FIG. 37 is an axonometric, plan view of a thermoformed syringe showing the die-cut sheath prior to thermoforming;

FIG. 38 is an axonometric, side view of the integral, thermoformed sheath of FIG. 35

FIG. 39 is an enlarged, cross-sectional, side view of the sheath in the transporting posture;

FIG. 40 is an axonometric, side view of the thermoformed syringe with a retracted sheath of FIG. 35

FIG. 41 is an enlarged, longitudinal, cross-sectional, detail view of the thermoformed syringe of FIG. 35 showing rotational interference of the retracted sheath;

FIG. 42 is a plan view of the sheath with a transporting cannula engagement;

FIG. 43 is a sectioned, front view of the sheath with a locking, disposal, cannula engagement;

FIG. 44 is a plan view of the sheath with a transporting cannula engagement

FIG. 45 is a sectioned, front view of the sheath with a locking, disposal, cannula engagement;

FIG. 46 is a longitudinal, side view of the thermoformed, tubular plunger with a frustoconical plug seal;

FIG. 47 is a longitudinal, cross-sectional, side view of the thermoformed, tubular plunger in FIG. 45;

FIG. 48 is an axonometric view of a syringe using heat shrink, extruded tubing in accordance with an alternative embodiment of the invention;

FIG. 49 is a longitudinal, cross-sectional, side view of the thermoformed syringe of FIG. 48;

FIG. 50 is an axonometric, side view of the thermoformed, tubular sheath;

FIG. 51 is an expanded axonometric, side view of the thermoformed, tubular plunger with a pierceable, sealing grommet and optional pierceable cap;

FIG. 52 is an expanded, axonometric, cross-sectional, side view of the thermoformed, tubular plunger from FIG. 51;

FIG. 53 is a cross sectional view of the plunger shaft revealing the central, access lumen, guiding fins, and lumen support members.

### DETAILED DESCRIPTION

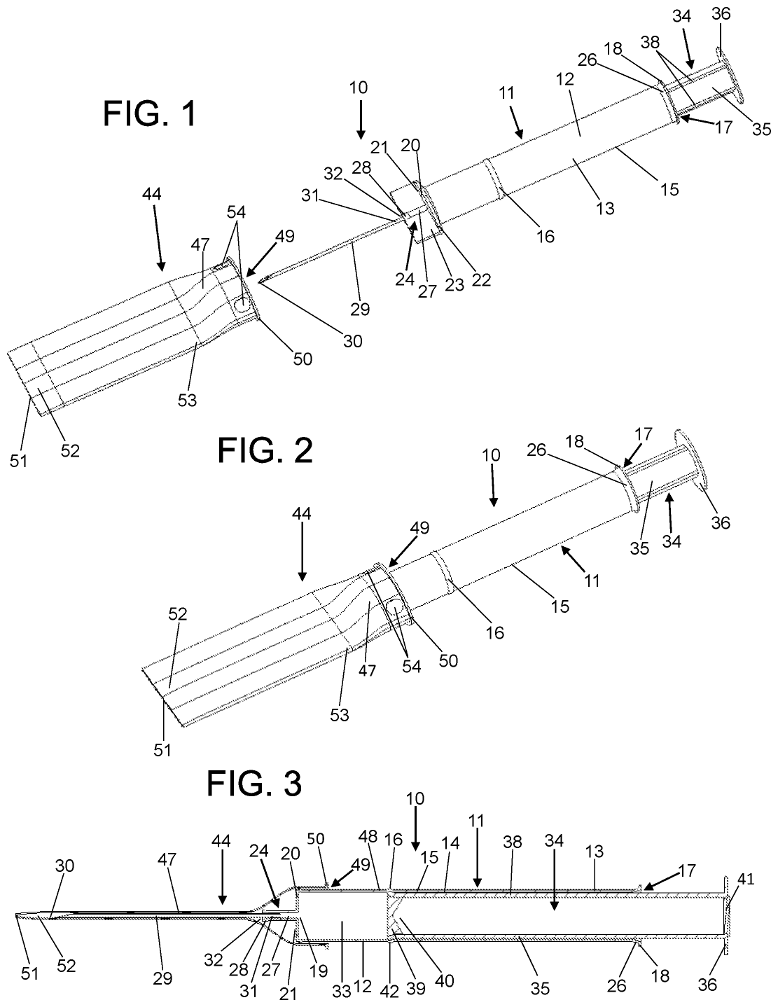

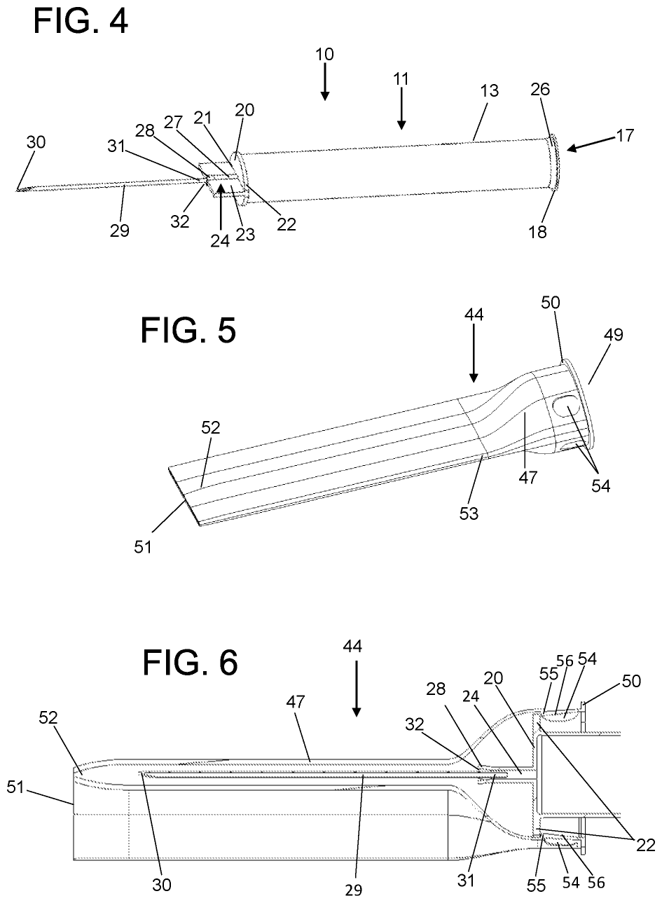

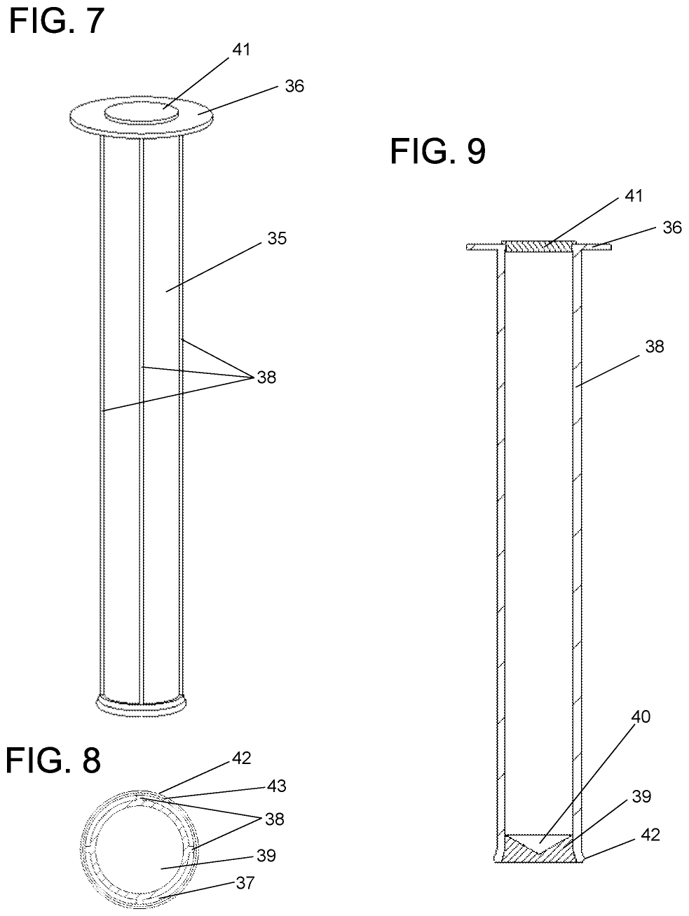

Referring now particularly to the drawings, wherein like reference characters refer to like parts, and initially to FIGS. 1, 2, and 3, there will be seen axonometric and cross-sectional views of a thermoformed syringe 10 in accordance with one preferred embodiment of the invention. The syringe 10 in FIGS. 1, 2, and 3 comprises a tubular body 11 having a flanged, proximal opening 17 and a truncated, thermally sealed distal end 19 with a linear, fluid passageway 24 within the sealing flange 23 fluidly communicating with an axially aligned cannula 29 (needle); and an axially sliding plunger 34 within a barrel 12 providing a sealing means to define a fluid chamber 33. The barrel 12 comprises a length of extruded tubing 13 with a uniform interior diameter 14 within a thin, resilient sidewall 15. A thermoformed, peripheral flange 18 of proximal opening 17 maintains the circular nature of the barrel 12 and assists holding the syringe 10 during filling and administering an injection. A slight flare 26 in sidewall 15 assists insertion of the plunger 34 prior to thermoforming end wall 20. The barrel 12's distal end 19 is thermoformed and sealed under slight, axial pressure over the plunger 34's seal ring 42 producing a smooth, truncated end wall 20 with a distal passageway 24 with a slight, distal flare 28. The seal 21 has a linear profile. Two, opposing, elliptical selvages 22 are formed flanking the distal end wall 20 as a result of the end wall's compression when sealing. They become the positioning posts for the cannula's sheath 44. To assemble the cannula, lateral compression is applied about the seal selvages 22 causing the neck 27 and distal flare 28 of the passageway 24 to open and receive the cannula 29. When released the neck portion 27 retracts and grips the cannula 29 and maintains the cannula's posture prior to being attached and sealed within the distal flair 28 with an applied adhesive 32. Now referring to FIGS. 7, 8 and 9, the plunger 34 is a thermoformed, extruded tube 35 with a distal, integral, fluid sealing flange 37 with occluding plug 39 and a proximal, integral, finger pad ring 36. The fluid seal is established by the resiliency of the barrel sidewall 15 and the seal interface 42 supported by rigid, plunger sealing flange 37. Integral, lateral, radiating fins 38 of the extruded, tubular shaft 35 ensure the plunger 34's axial travel within the tubular body 11 maintaining its fluid tight seal with the resilient, barrel sidewall 15. The distention 16 occurring in the barrel 12's resilient sidewall 15 with plunger 34's travels is caused by the dimensional interference of the sealing flange 37's interface 42 and the barrel 12's interior diameter 14. This is the basis of the fluid tight seal. FIG. 4 reveals a barrel 12's smooth sidewall 15 without an inserted plunger 34. The cannula 29 has one end 30 sufficiently sharp to pierce the patient's skin and the other end 31 blunt to receive the affixing adhesive 32. FIGS. 2 and 5 reveal a cannula sheath 44 of extruded tubing 47 with a circumference 48 sufficient to pass over the syringe body 11's seal selvage 22. A thermal seal 51 closes the distal end 52 and partially wraps the sides 53 to restrict its axial travel onto the syringe 10 protecting the cannula tip 30. Toward the proximal end 49, four, opposing, transverse indentions 54 engage the flanged, selvage section 22 for securement. To assemble, the sheath 44 is axially slid onto the syringe body 11until the indentions 54 engage the selvage 22. To remove, simply reverse the procedure.

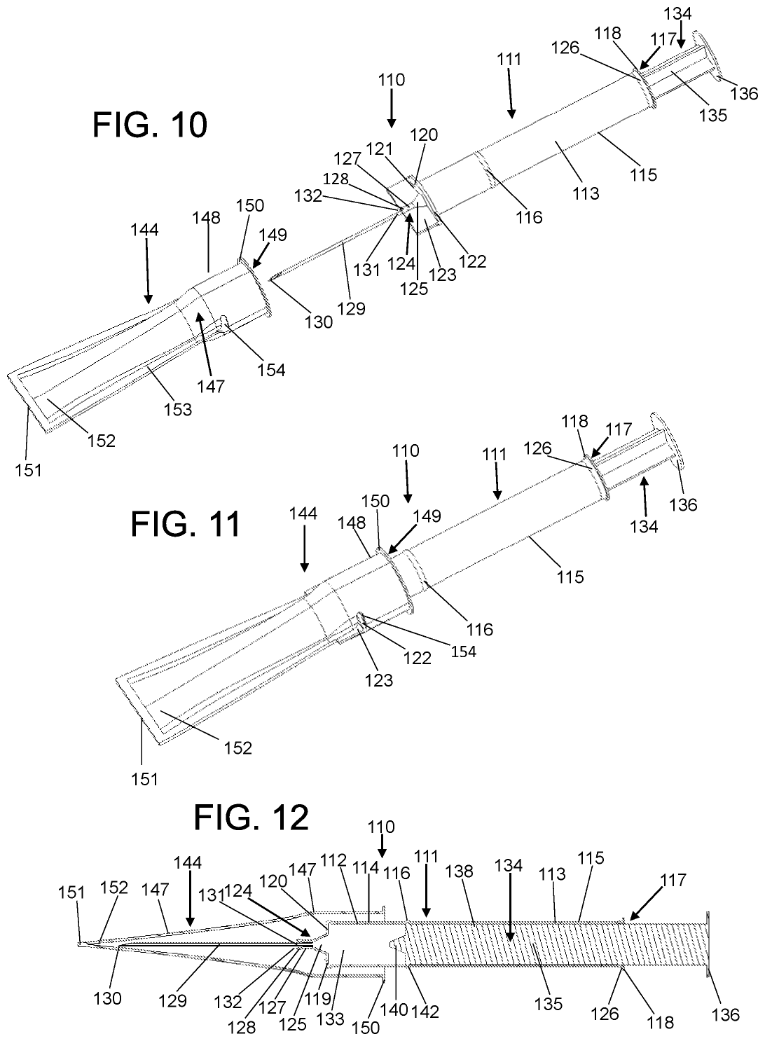

The syringe 110 in FIGS. 10, 11, and 12 comprises a tubular body 111 having a flanged, proximal opening 117 and a truncated, thermally sealed distal end 119 with a nonlinear, fluid passageway 124 through the sealing flange 123 fluidly communicating with an axially aligned cannula 129 (needle); and an axially sliding plunger 134 with a sealing interface 142 to define a fluid chamber 133 within barrel 112. The barrel 112 comprises a length of extruded tubing 113 with a uniform, interior diameter 114 and a thin, resilient sidewall 115. The thermoformed, peripheral flange 118 of proximal opening 117 maintains the circular nature of the barrel 112 and assists holding the syringe 110 during filling and administering an injection. A slight flare 26 in sidewall 15 assists insertion of the plunger 34 prior to thermoforming end wall 20. The barrel's distal end 119 is thermoformed and sealed under slight, axial pressure over the plunger 134's seal disc 139 with a central, frustoconical stanchion 140 producing a smooth, end wall 120 with a distal, frustoconical passageway 124. The barrel seal 121 has a nonlinear profile creating a more rigid, canular posture within end wall 120. Two, opposing, elliptical selvages 122 are formed flanking the distal end wall 120 as a result of the end wall compression when sealing. They become the positioning posts for the cannula's sheath 144. To assemble the cannula 129 into the body 111, lateral compression is applied about the seal selvage 122 and seal flange 123 with frustum 125 to initiate the opening of the distal flare 128 of the passageway 124 to receive the cannula 129. When released the neck portion 127 retracts and grips the cannula 129 to maintain the cannula's posture prior to being attached and sealed within the distal flare 128 with an applied adhesive 132. Now referring to FIGS. 15 and 16, the plunger 134 is injection molded with a central, cruciform shaft 135 supporting opposing, integral discs: proximally a finger pad 136 and distally a seal 139. The cruciform legs 138 within the tubular body 111 ensure the normal, axial travel of the sealing disc 139. The seal disc interface 142 must be free of surface irregularities, like a parting line 143. This is accomplished with a mold core undercut in the B-side of the mold base forming the seal interface 142 detail. When ejected, the sealing interface 142 and supporting seal disc 139 slightly compress to overcome the undercut and the plunger 134 tumbles from the B-side mold base.

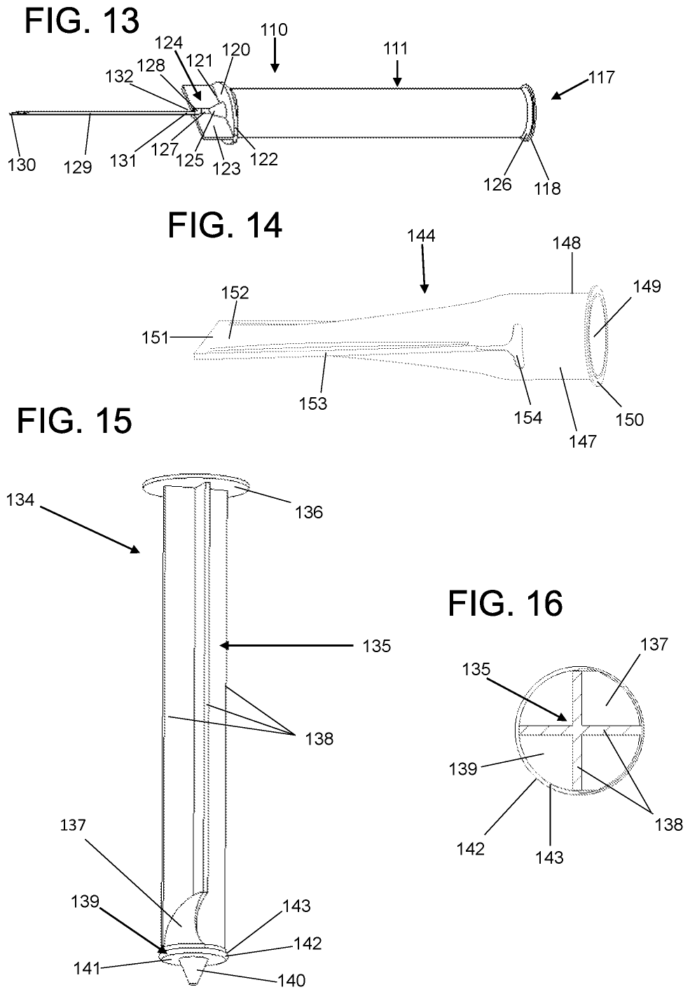

The distention 116 occurring in the barrel's, resilient sidewall 115 with plunger 134 movement is caused by the dimensional interference of the sealing disc 139's interface 142 and the barrel 112's interior diameter 114. This is the basis of the fluid tight seal. FIG. 13 reveals a smooth, body 111 sidewall 115 when the plunger 134 is not inserted into the barrel 112. The cannula 129 has one end 130 sufficiently sharp to pierce the patient's skin and the other end 131 blunt to receive the affixing adhesive 132. FIGS. 10 and 14 reveal a cannula sheath 144 of extruded tubing 147 with a circumference 148 sufficient to pass over the syringe body's 111 seal selvage 122. Peripheral flange 150 strengthens the sheath 144's proximal opening 149 to further secure its attachment. Thermal seal 151 closes the distal end 152 and partially wraps the sides 153 to restrict its axial travel onto the syringe 110 protecting the cannula tip 130. Toward the proximal end 149 are two, opposing, transverse apertures 154 which engage the flanged, selvage section 122. To assemble, the sheath 144 is slightly compressed about the top and bottom to clear the body's sealing, selvage 122 as it's axially slid onto the syringe body 111, and then released for the side-apertures 154 to engage the selvage 122 for attachment. To remove, simply reverse the procedure.

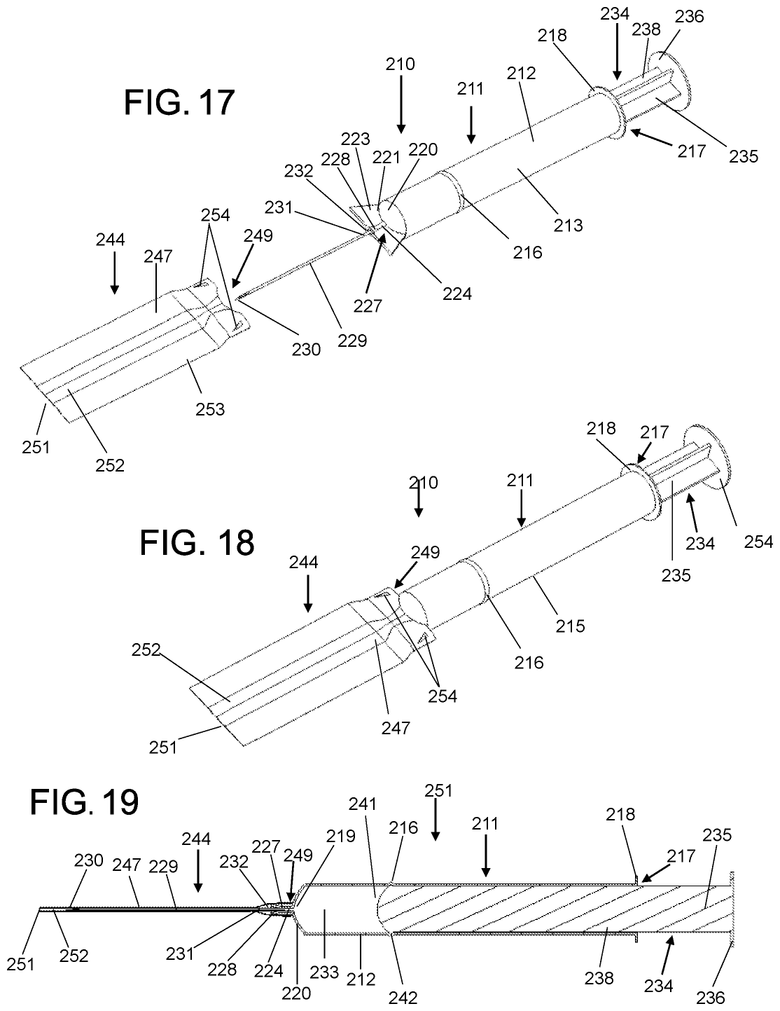

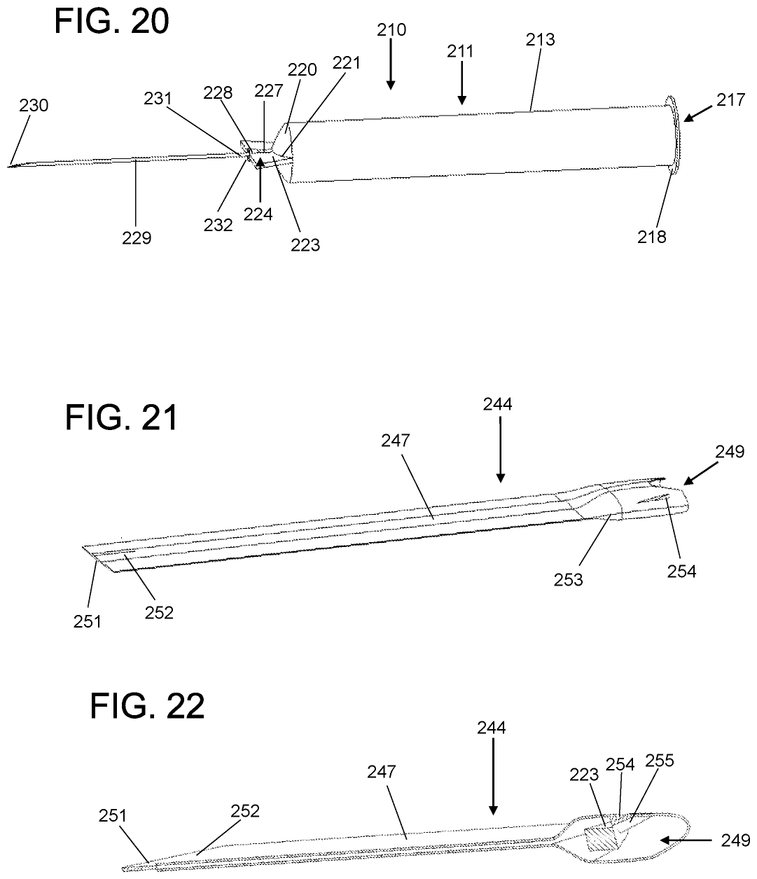

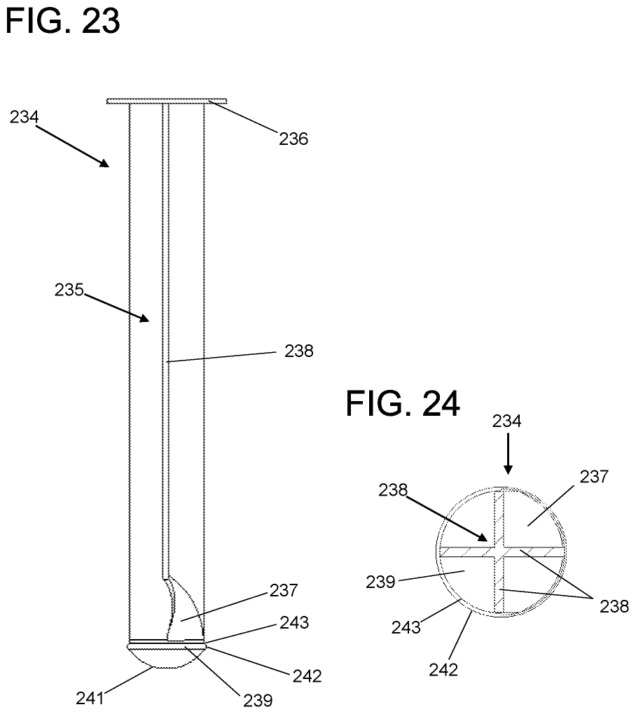

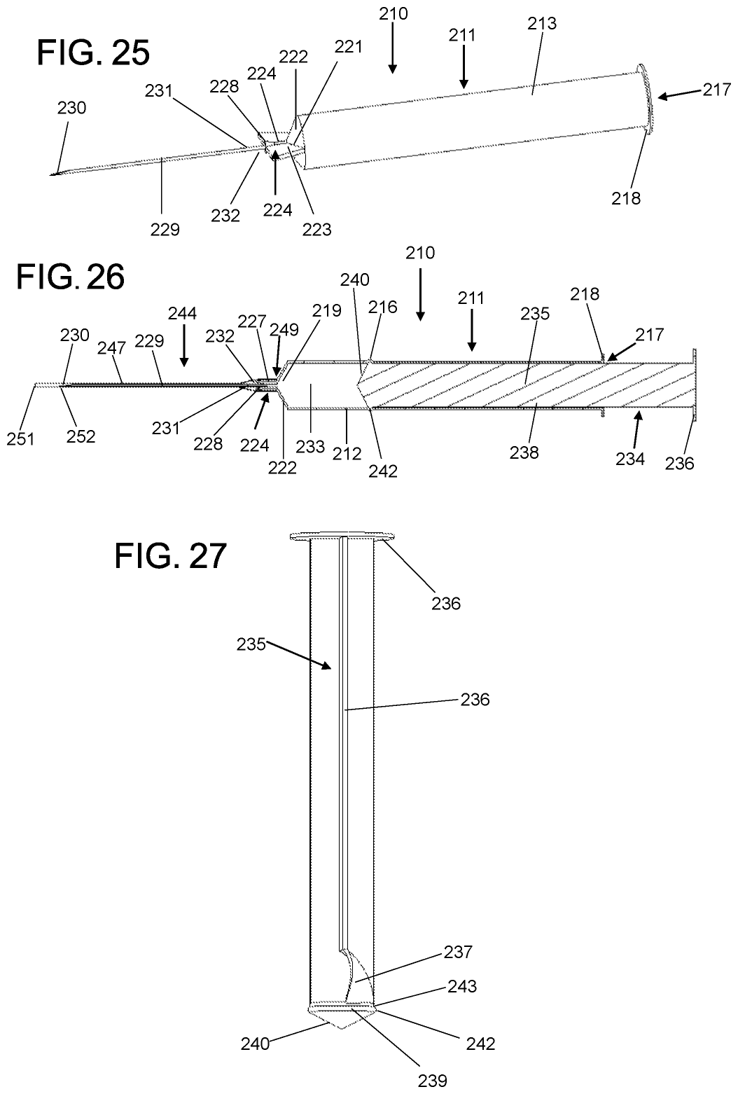

The syringe 210 in FIGS. 17, 18, 19, 23, 24, and 25 comprises a resilient, tubular body 211 having a flanged, proximal opening 217 and a domed, thermally sealed distal end 219 with a linear, fluid passageway 224 through the sealing flange 223 fluidly communicating with an axially aligned cannula 229 (needle); and an axially sliding plunger 234 with a sealing means 239 within barrel 212 to define a fluid chamber 233. The barrel 212 comprises a length of extruded tubing 213 with a uniform interior diameter 214 within a thin, resilient sidewall 215. A thermoformed, peripheral flange 218 of proximal opening 217 maintains the circular nature of the barrel 212 and assists holding the syringe 210 during filling and administering an injection. The domed, distal end wall 220 consists of two conjoined, circular parabolic sections; or the conic, distal end wall 222 consists of two, conjoined, conic sections. These result from being axially sealed over the inserted, molded plunger 234's distal sealing member 239 with a distal extension 241 or 240, respectively, of matching profile for full medicant evacuation. The linear seal 221 is interrupted to provide a liquid passageway 224 through the seal flange 223 with a distal, adhesive flair 228. For assembly, the cannula 229 is partially inserted into the passageway 224 with slight lateral compression about the seal flange 223 and secured with adhesive 232. Now referring to FIGS. 23 and 24, the plunger 234 is injection molded with a central, cruciform shaft 235 supporting opposing, integral discs: proximally a finger pad 236 and distally a seal 239. The cruciform legs 238 within the tubular body 211 ensure the normal, axial travel of the sealing disc 239. The seal disc interface 242 must be free of surface irregularities, like a parting line 243. This is accomplished with a mold core undercut in the B-side of the mold base forming the seal interface 242 detail. When ejected, the sealing interface 242 and supporting seal disc 239 slightly compress to overcome the undercut and the plunger 234 tumbles from the B-side mold base. The fluid seal is established by the resiliency of the barrel sidewall 215 and the seal interface 242 supported by rigid, plunger sealing flange 239. The distention 216 occurring in the barrel 212's, resilient sidewall 215 with plunger 234's travels is caused by the dimensional interference of the sealing flange 237's interface 242 and the barrel 212's interior diameter 214. FIG. 20 reveals a barrel 212's, smooth sidewall 215 without an inserted plunger 234. The cannula 229 has one end 230 sufficiently sharp to pierce the patient's skin and the other end 231 blunt to receive the affixing adhesive 232. FIGS. 21 and 22 reveal a cannula sheath 244 of extruded tubing 247. A thermal seal 251 closes the distal end 252 and partially wraps the sides 253 to restrict its axial travel onto the syringe 210 protecting the cannula tip 230. Toward the proximal end 249, two, opposing, transverse indentions 254 engage the seal flange 223 for securement. To assemble, the sheath 244 is axially slid onto the syringe body 211 until the indention 254's sloped surfaces 255 engage the selvage 222. To remove, simply reverse the procedure.

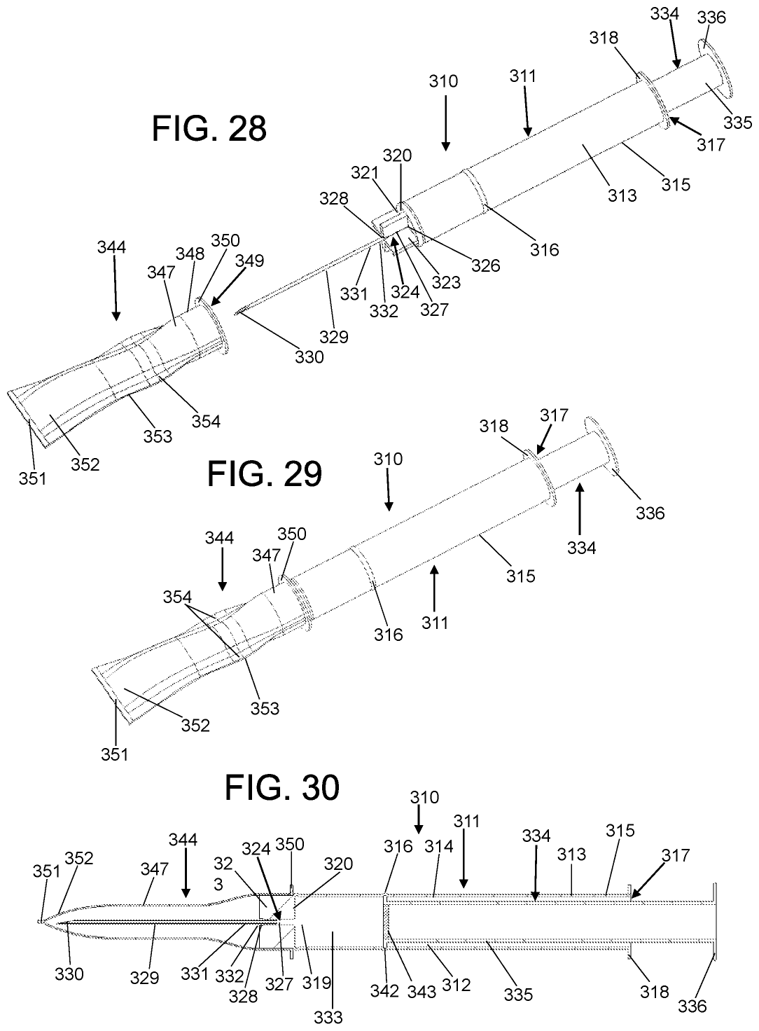

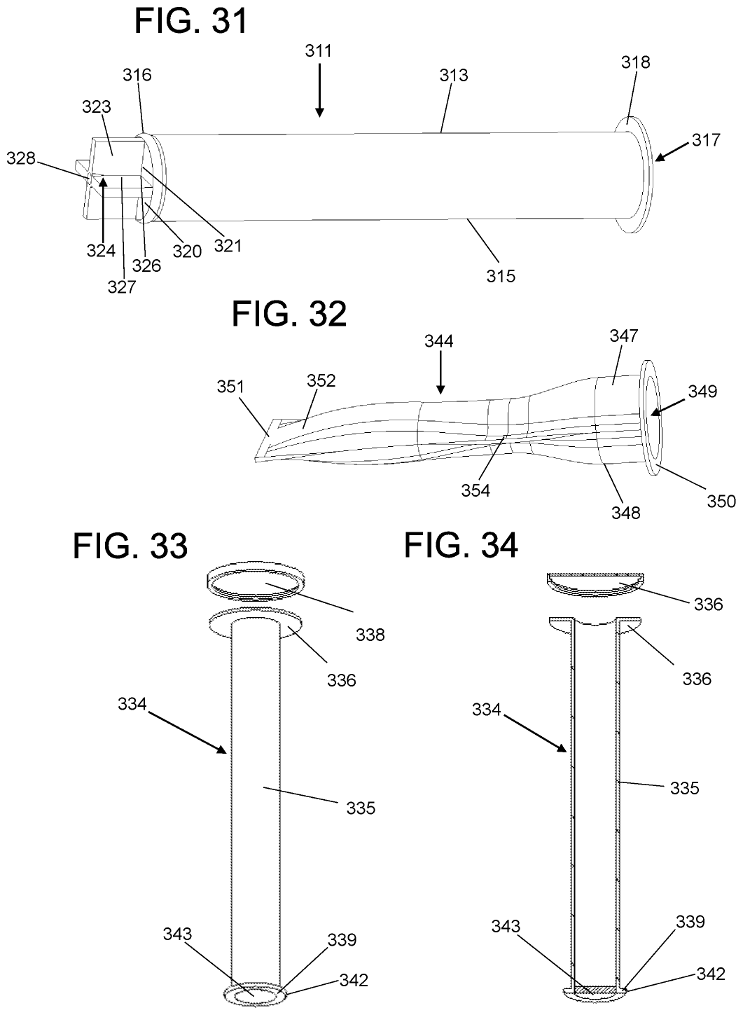

FIGS. 28, 29, and 30 reveal another embodiment 310 of a syringe comprising of an extruded, tubular body 311 having a flanged, proximal opening 317, a closed distal end 319 with a passageway 324, fluidly communicating with an axially aligned cannula 329 (needle), and an axially sliding, sealing plunger 334 establishing a liquid chamber 333 within the barrel 312. The barrel 312 comprises a length of extruded tubing 313 with a uniform, interior diameter 314 and a thin, resilient sidewall 315. A thermoformed, peripheral flange 318 of proximal opening 317 maintains the circular nature of the barrel 312 and assists holding the syringe 310 during filling and administering an injection. The distal end 319 of the extruded tube 313 is folded into the plunger 334's distal surface 341 forming indentions 326 and producing multiple, nonplanar, distal flanges 323 which diminish the seal selvage; and is sealed about the base 321 of the seal flanges 323. This distal structure creates a rigid end wall 320 for cannular posturing with a linear, fluid passageway 324 having a slight, distal flare 328. To assemble the cannula 329 into the body 311, axial pressure is applied to the plunger 334 forcing the seal flanges 323 to move distally causing the neck 327 of passageway 324 to open and receive the cannula 329. When released the neck portion grips the cannula and maintains its posture prior to being attached and sealed within the passageway's distal flair 328 with an applied adhesive 332. The cannula 329 has one end 330 sufficiently sharp to pierce the patient's skin and the other end 331 blunt to receive the affixing adhesive 332. FIGS. 33 and 34 show a plunger 333 with a tubular shaft 335 with flanged ends 336 and 339. The proximal flange 336 exceeds the width of the barrel's flange 318 for accessibility and becomes the finger pad 336. The distal flange 339 becomes the sealing interface 342 establishing the fluid-tight seal with the barrel 312's resilient sidewall 315. The distention 316 occurring in the barrel's resilient sidewall 315 with plunger 334's movement is caused by the dimensional interference of the interface 342 and the interior diameter 314. This is the basis of the fluid tight seal. An occluding plug 343 is pressure fit into the distal end of shaft tube 335. The occluding plug 243 caps said shaft tube 235 with a distal surface mimicking the end wall 320 contour and becoming the sealing interface with the body sidewalls. A pierceable cap 338 may cover the proximal opening 317 for sealed access. Without a plunger 334 inserted into barrel 312, a smooth sidewall 315 results and is shown in FIG. 31. FIG. 32 reveal the new, needle sheath 344 also thermoformed from a section of extruded tubing 347 with a circumference 345 sufficient to frictionally fit over the distal flanges 323. A thermal seal 351 closes the distal end 352. A pair of opposing, spot seals 354 are placed on the sheath's sidewall 353 to restrict its axial travel onto the syringe 310 protecting the cannula tip 330.

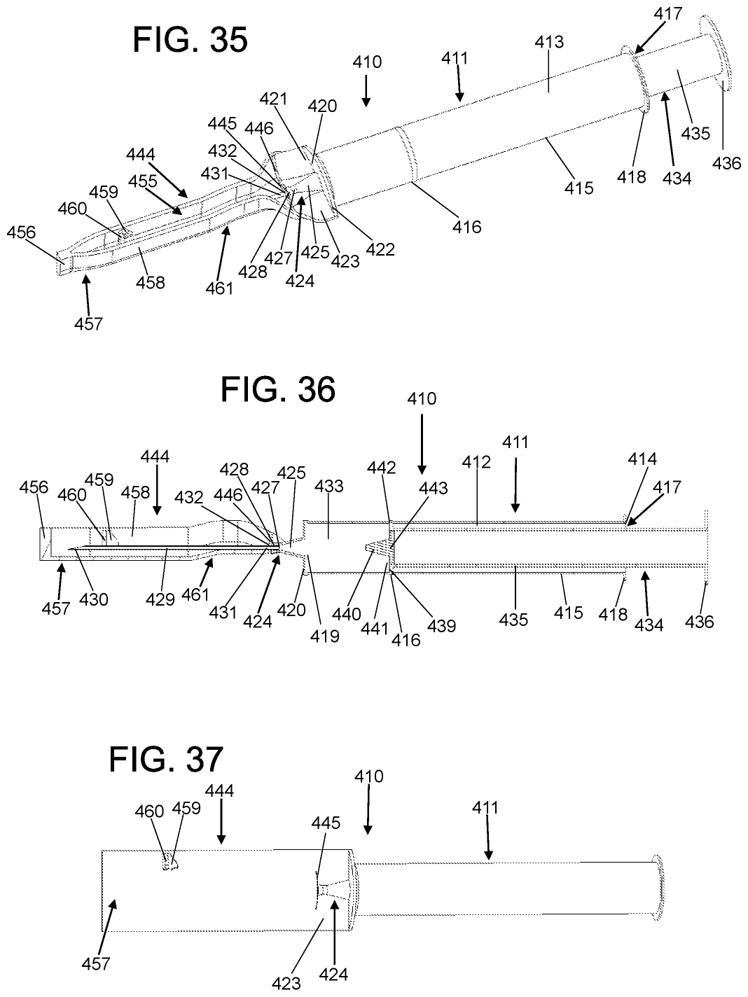

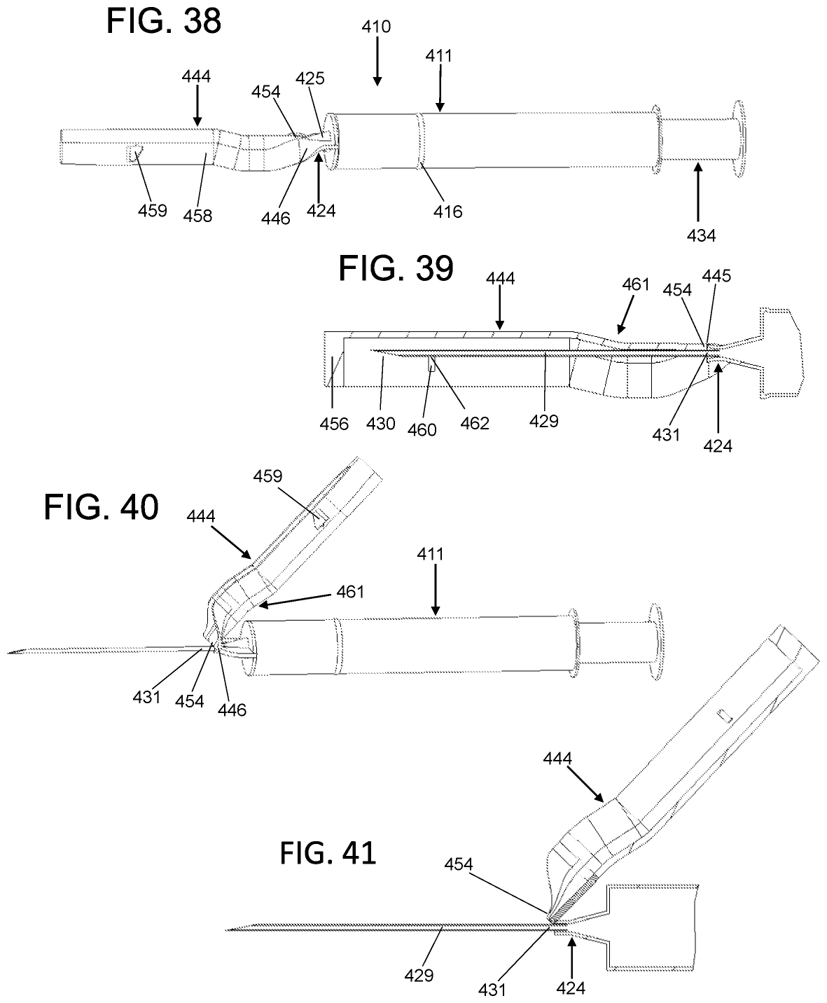

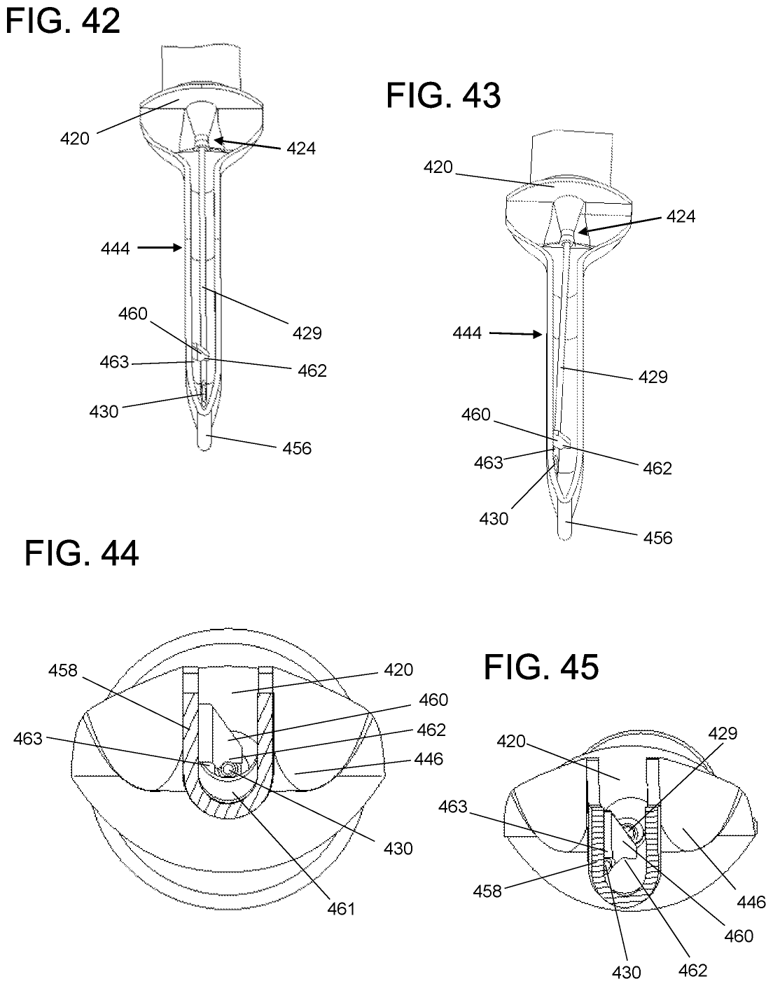

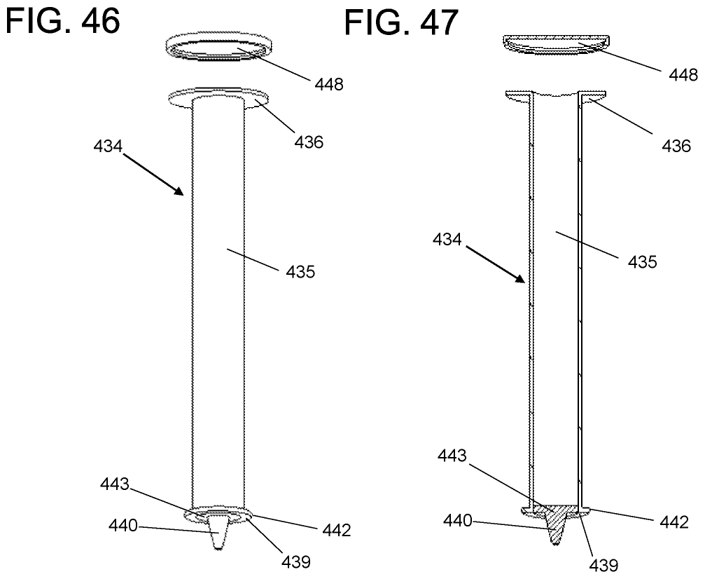

FIG. 35 is an axonometric view and FIG. 36 is a longitudinal cross-sectional, side view of another embodiment of a thermoformed syringe 410 with the body 411 having an integral, selectively rotating, cannula (needle) sheath 444. The syringe's tubular body 411 is thermoformed using extruded tubing 413 providing a uniform, interior diameter 414 within a thin, resilient sidewall 415. The body 411 comprises a barrel 412 with a flanged, proximal opening 417, an intermediary sealed end 419 with a stepped, frustoconical passageway 424 distally accepting a cannula 429, and a distal, integral, selectively rotating sheath 444. At the open, proximal end 417 of barrel 412, a thermoformed, peripheral gripping flange 418 maintains the circular nature of the barrel 412 and assists the holding of the syringe 410 during filling and administering an injection. The intermediary sealed, wall 420 is thermoformed and sealed over the plunger 434's seal disc 439's frustoconical stanchion mimicking the disc's distal geometry. The resulting, distal, frustoconical stepped passageway 424 stiffens the barrel 412's end wall 420 and provides visual, fluid communication between the fluid chamber 433 and a cannula 429. As a result of this sealing process with slight axial pressure, two elliptical selvage regions 422 and distal flange 423 are formed flanking and extending from the distal seal 421. FIG. 37 reveals the planar flange 423 is arcuately die-cut 445 distally beyond the frustum 425 and thermoformed slightly downward into a selectively rotating, trough sheath 344. FIGS. 38 and 40 reveal the sheath's attachment sections are living hinges 446 providing selective posturing of the sheath 443 by a rotating interference between the proximal, central trough region 455 and the cannula base 431 due to arcuate cut 445. FIGS. 38, 39, 40, and 41 reveal that as the sheath 444 rotates, the interference causes a lengthening and distortion of the living hinges 446. Once over center, the interference lessens and the living hinges 446 retract, selectively posturing the sheath 444. To assemble the cannula, the sheath 444 is rotated to its stowed position to expose the distal flare 428 and lateral compression is applied about the seal selvage 422 causing neck 427 and distal flare 428 of the passageway 424 to open and receive the cannula 429. When released the neck portion 427 retracts and grips the cannula 429 maintaining the cannula's posture prior to being attached and sealed within the distal flair 428 with an applied adhesive 432. The cannula 429 has one end 430 sufficiently sharp to pierce the patient's skin and the other end 431 blunt to receive the affixing adhesive 432. FIGS. 42, 43, 44, and 45 demonstrate the sheath 444 and cannula 429 engagement for transporting and disposal of the syringe 410. In the sheath's sidewall length 458, tab 460 is inwardly formed with two slots 462 and 463 of sufficient depth to engage the cannula 429. The trough has an intermediate, downward offset 461 to separate the engagement intentions of the individual slots 462 and 463. Slot 462 selectively engages the cannula 429 for safe transporting and slot 463 locks over the cannula 429 for secure, syringe disposal. Locking requires an intentional, outward distortion of the trough sidewalls 458 for the locking slot 463 to engage the cannula 429. The trough distal end 457 supports seal 456 to prevent axial exposure of cannula tip 430. FIGS. 46 and 47 show a plunger 434 with a tubular shaft 435 with flanged ends 436 and 439. The proximal flange 436 exceeds the width of the barrel flange 418 for accessibility and becomes the finger pad 436. The distal flange 439 becomes the sealing interface 442 establishing the fluid-tight seal with the barrel 412's resilient sidewall 415. The distention 316 occurring in the barrel's resilient sidewall 415 with plunger 434's movement is caused by the dimensional interference of the interface 442 and the interior diameter 414. This is the basis of the fluid tight seal. A pierceable, occluding plug 443 is pressure fit into the distal end of shaft tube 435. A pierceable cap 448 may cover the proximal opening 447 for sealed access.

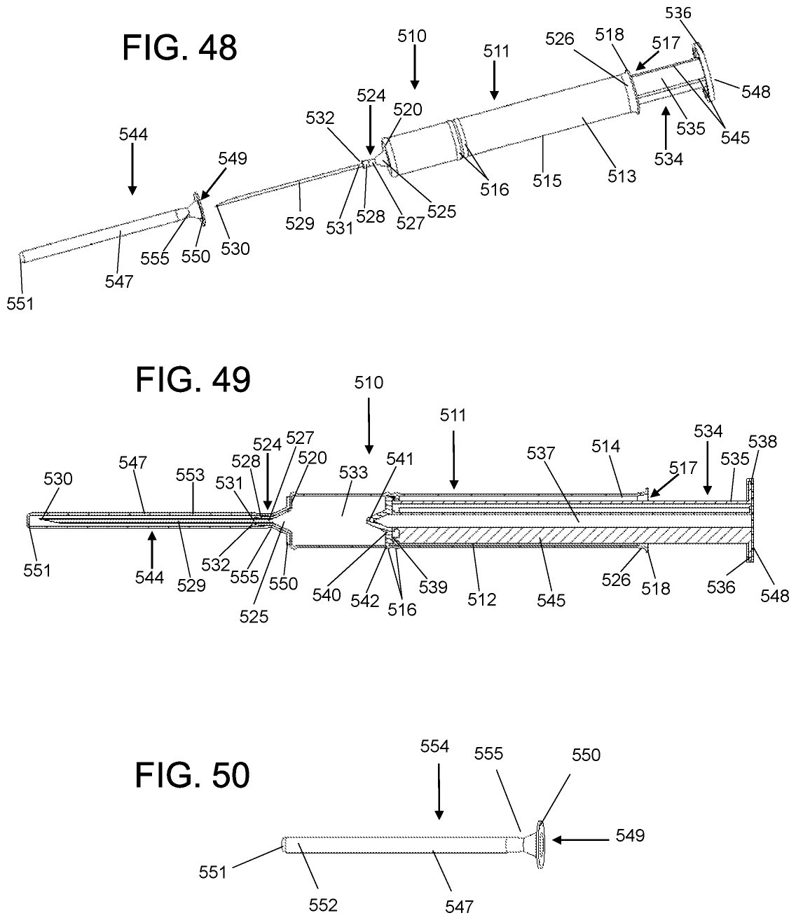

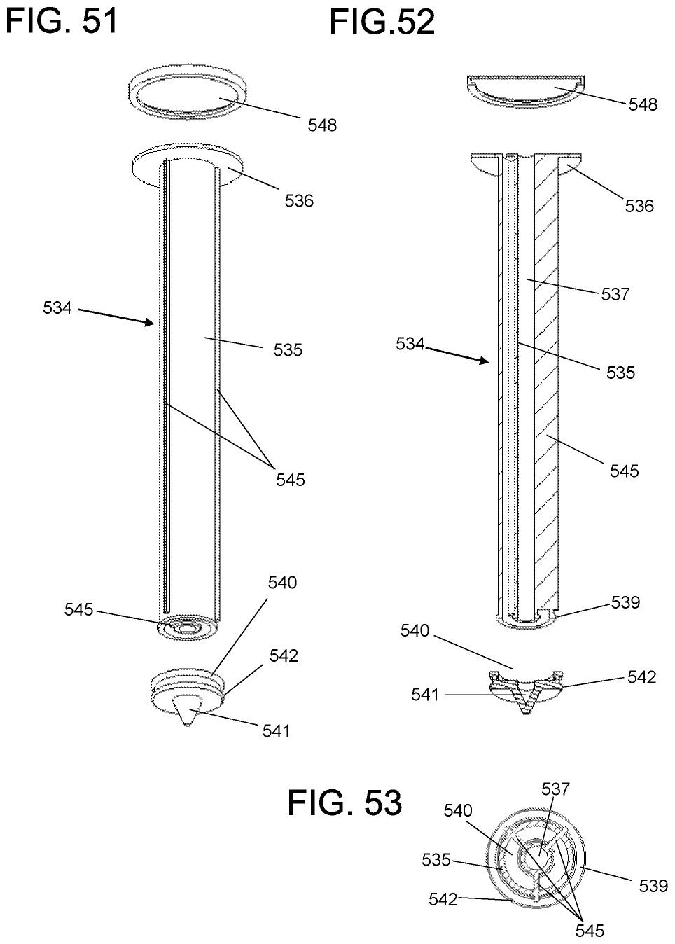

FIG. 48 is an axonometric view and FIG. 49 is a longitudinal cross-sectional, side view of another embodiment of a syringe 510 using extruded, heat shrink tubing 513 to form the body 511. The tubular body 511 has a uniform diameter 514 within a thin, resilient sidewall 515. Its open, proximal end 517 is peripherally flanged 518 for rigidity and gripping ease when holding the syringe 510 during filling and administering an injection. The distal end 519 is closed by heat shrinking over the circular, seal interfaces 542 of the grommet 540 supported by the sealing flange 539. Slightly up on its base, the cannula 529 is held within the tapering tip of a positioning fixture while the tube 513 is heat shrank around the plunger 534's seal flange 539's interface 543, the frustoconical, capping grommet 540, cannula base 531, and tapering tip. This produces an end wall 520 with a frustum 525 having a distal neck 527 posturing the cannula 529, and an adhesive/sealer reservoir 528 created by the retraction of the fixturing tip. The cannula 529 is affixed by placing an adhesive/sealer 532 in the reservoir 528. The distention 516 occurring in the barrel's, resilient sidewall 515 with plunger 534's movement is caused by the dimensional interference of the grommet 569's sealing interface 542 and the barrel 512's interior diameter 514. This is the basis of the fluid tight seal. FIGS. 51, 52 and 53 show a multi-lumen plunger 534 using an extruded tubular shaft 535 with center lumen 537, flanged ends 538 and 539, a perforable, distal, frustoconical, capping grommet 540, a proximal, perforable cap 548, at least three radiating, guide/stabilizing fins 545 ensuring axial, plunger travel. The proximal flange 538 with cap 548 becomes the finger pad 536 and exceed the width of the barrel flange 518 for accessibility and the grommet 540 supported by the distal flange 539 establishes a fluid-tight seal with the barrel's resilient sidewall 515. FIG. 50 reveals a new, cannular sheath 544 of heat shrink, extruded tubing 547. The open, proximal end 549 is heat shrank over the frustum 525 and the flared, adhesive reservoir 528, and the distal end 552 is heat shrank closed for cannular protection.

What is claimed is:

1. A syringe comprising:
a tube means having a resilient side wall with an open proximal end and a sealed, distal end fluidly communicating via a lumen through seal;
a distal intravenous cannula; and
a plunger means having a shaft supporting a distal, fluid sealing means and a finger pad at opposite ends moveably disposed within said tube.

2. A syringe as defined in claim 1 and further comprising:
a side wall resiliency resulting from material selection and thinness translating to manufacturing efficiencies, material economies, and sustainability.

3. A syringe as defined in claim 1 and further comprising:
said thermal truncation and sealing of distal end wall with slight axial compression create both a pair of selvage regions flanking barrel end wall and a distal flange beyond seal shaping the fluid lumen.

4. A syringe as defined in claim 1 and further comprising:
said plunger having an efficient, tubular shaft supporting a distal flange with an unblemished interface establishing a fluid tight seal with resilient sidewall.

5. A syringe as defined in claim 1 and further comprising:
a flange about said open, proximal, barrel end for rigidity and handling ease.

6. A syringe as defined in claim 1 and further comprising:
a pierceable, distal plug occluding said plunger tubular shaft permitting patient procedural access.

7. A syringe as defined in claim 1 and further comprising:
a volume indicia printed on said, barrel side wall to be referenced by the plunger seal position within barrel.

8. A syringe as defined in claim 1 and further comprising:
a sheath means for said cannula distally sealed for tip protection, having side detents abutting seal flange to limit axial travel onto syringe, and proximal engagement indentions for selective securement to seal selvage.

9. A syringe as defined in claim 1 and further comprising:
said fluid lumen means having a frustoconical profile for cannular stability and aspirate viewing.

10. A syringe as defined in claim 1 and further comprising:
said plunger means having a cruciform shaft supporting a distal sealing disc with an unblemished interface establishing fluid seal with resilient sidewall and a proximal finger pad.

11. A syringe as defined in claim 1 and further comprising:
a said sheath means for said cannula distally sealed for tip protection, having side detents abutting seal flange to limit axial travel onto syringe, and proximal, side, engagement apertures for selective securement to the seal selvage.

12. A syringe as defined in claim 1 and further comprising:
said barrel seal is arcuate creating a simple, domed end within barrel's circumference producing a paraboloidal extension of the barrel for cannular posture stability.

13. A syringe as defined in claim 1 and further comprising:
said sealed end is linearly tapered within barrel's circumference producing a conical extension of the barrel for cannular posture stability.

14. A syringe as defined in claim 1 and further comprising:
a said sheath for said cannula with a sealed distal end for tip protection, side detents limiting axial travel onto syringe, and proximal engagement indentions for selective securement to the tapering, distal, seal flange.

15. A syringe as defined in claim 1 and further comprising:
a nonplanar end wall seal with indentions for cannular posture stability and to lessen seal selvage.

16. A syringe as defined in claim 1 and further comprising:
perforable plug/cap occluding proximal opening of said tubular shaft.

17. A syringe as defined in claim 1 and further comprising:
said plunger, fluid sealing flange means supports an elastomeric grommet to establish fluid tight engagement with barrel sidewalls and provide sealed, barrel access.

18. A tube as defined in claim 1 and further comprising:
said plunger, tubular shaft means with multiple, lateral lumens for sealed, patient access through syringe barrel.

19. A syringe as defined in claim 1 and further comprising:
said tubular shaft means with multiple, exterior, radiating, guiding fins ensuring axial, efficient plunger travel.

20. A syringe as defined in claim 1 and further comprising:
said body tube means of heat shrink material for an efficient forming process.

21. A syringe comprising:
a tube means of resilient material having an open proximal end and an intermittent seal fluidly communicating via a stepped lumen;
an integral, distal sheath pivoting for cannular access and cannular shielding or locking postures;
a distal intravenous cannula; and
a plunger shaft means being tubular for efficiency, material economy, and selective, patient access through perforable grommet, moveably disposed within said tube.

22. A syringe as defined in claim 21 and further comprising:
said sealed, barrel end is truncated with peripheral selvage allowing planar edge reference between sealing means and the printed, volume indicia.

23. A syringe as defined in claim 21 and further comprising:
said lumen is frustoconical for cannular posture stability and viewing concentrated aspirate.

24. A syringe as defined in claim 21 and further comprising:
a perforable, occluding plug with a distal surface mimicking the end wall contour supports said fluid sealing ring to ensure its circular configuration, total medicant evacuation, and sealed patient access.

25. A syringe as defined in claim 21and further comprising:
said plunger's distal flange supporting an elastomeric grommet with a distal surface mimicking the end wall contour to establish fluid tight engagement with barrel sidewalls, total medicant evacuation, and provide sealed, barrel access.

26. A syringe as defined in claim 21 and further comprising:
said plunger shaft with multiple lumens for appliance conduits with sealed access to syringe barrel.

27. A syringe as defined in claim 21 and further comprising:
a flange about said open, proximal barrel end for rigidity and handling ease.

28. A syringe as defined in claim 21 and further comprising:
said body fluid lumen of sufficient length to view aspirate leaving cannula and entering barrel.
